(19) United States
(12) Patent Application Publication
(10) Pub. No.: US 2026/0328757 A1
(43) Pub. Date: Oct. 22, 2026

(54) SYSTEMS AND METHODS FOR ANALYTE DETECTION

(71) Applicant: ONE LAMBDA, INC., West Hills, CA (US)

(72) Inventors: Rui PEI, West Hills, CA (US); Ito WAKEFIELD, Sherman Oaks, CA (US); Hong MA, Oak Park, CA (US)

(21) Appl. No.: 19/468,849

(22) Filed: Feb. 3, 2026

Related U.S. Application Data
(63) Continuation of application No. PCT/US2024/043260, filed on Aug. 21, 2024.
(60) Provisional application No. 63/578,341, filed on Aug. 23, 2023.

(51) Int. Cl. G01N 33/53; G01N 33/543; G01N 33/58

(52) U.S. Cl. CPC G01N 33/5308; G01N 33/54388; G01N 33/585; G01N 2333/522; G01N 2333/9015; G01N 2333/9126

(57) ABSTRACT

Methods, systems, and devices for analyzing a sample for the presence of IP-10 are provided. A sample can be processed, the processing generating an amplification product of a template nucleic acid formed in response to a target protein being present in the sample. The amplification product can be contacted with a detection probe to generate a second product comprising a labeled nucleic acid. A lateral flow substrate can be contacted with the second product, the lateral flow substrate comprising a capture moiety configured to bind the labeled nucleic acid. The methods can be carried out in one or more chambers of a single device or multiple devices. Detection probe bound to amplicons of the template nucleic acid captured on the lateral flow substrate can be read visually or using a sensor.

### CROSS REFERENCE TO RELATED APPLICATIONS

This application is a continuation of PCT application number PCT/US2024/043260, filed Aug. 21, 2024, which claims benefit of U.S. Provisional Application No. 63/578,341, filed Aug. 23, 2023. The entire contents of each of the foregoing applications are hereby incorporated by reference in their entirety.

### INTRODUCTION

Detection of an analyte such as a target protein present in a low amount, whether due to overall sample size or otherwise low concentration in a sample, poses challenging problems. For example, in some cases, intrinsic concentration of an analyte in a sample can fail to yield a product suitable for diagnostic processing. Further, processing the sample to achieve a suitable concentration for detection can require relatively laborious procedures. Moreover, sample volume can be limited so as to prohibit consideration of laborious sample concentration procedures.

In addition, because of the low sample volumes generally associated with “point-of-care” techniques, protein detection poses even greater challenges due to the sensitivities that can be needed to achieve accurate diagnostic indications. For example, use of lateral flow substrates and technologies to bind target proteins has faced difficulty in achieving the sensitivities needed for effective and accurate detection.

Accordingly, a need exists for methods, systems, and devices that can solve or otherwise ameliorate such problems associated with detection of proteins.

### BRIEF DESCRIPTION OF THE DRAWINGS

The accompanying drawings, which are incorporated into and form a part of the specification, illustrate embodiments of the present disclosure and, together with the description, serve to explain the principles of the disclosure. The drawings are only for the purpose of illustrating some embodiments of the disclosure and are not to be construed as limiting the disclosure. In the drawings:

FIG. 1 illustrates generally a work flow process for performing a protein detection assay in accordance with various embodiments of the present disclosure.

FIG. 2 illustrates schematically the work flow process for performing a protein detection assay of FIG. 1 in accordance with various embodiments of the present disclosure.

FIG. 3A through FIG. 3C are schematic diagrams that generally illustrate of stages of a proximity assay in accordance with various embodiments the present disclosure.

FIG. 4 illustrates generally a lateral flow device of the present disclosure.

FIG. 5 is a front perspective view that illustrates generally an instrument for receiving a lateral flow device of the present disclosure and carrying out an analysis.

### DETAILED DESCRIPTION

### Protein Analysis in Lateral Flow Devices and Systems

Various embodiments in accordance with the present disclosure pertain to methods, systems, and devices for detection of interferon gamma-induced protein 10 (IP-10) using proximity ligation assay (PLA) on a lateral flow (LF) platform. More specifically, methods, systems, and devices are disclosed that provide relatively high sensitivity to such detection so as to be able to determine the presence of the target analyte at relatively low concentrations and/or in relatively small sample volumes. Various embodiments of the present disclosure enable the use of a lateral flow substrate for detection of target protein in a sample. The ability to achieve such detection is unexpected in view of the difficulty heretofore encountered in being able to achieve sensitivities high enough to achieve such detection. To achieve such sensitivity when using a lateral flow substrate for detection, embodiments in accordance with the present disclosure utilize a target-linked template nucleic acid generation technique, such as occurs in a proximity assay, to first generate a template nucleic acid from a complex generated from the target protein of interest and amplify the template nucleic acid for subsequent detection on a lateral flow substrate. In this way, detection of the template nucleic acid serves as a proxy for detection of the target protein and can confirm the presence of the initial protein in the sample.

The terms “analyte” is to be construed broadly as any compound, molecule, or other substance of interest to be detected, identified, or characterized. For example, the analyte can include, but is not limited to, at least one of amino acids, nucleic acids, polypeptides, proteins, polynucleotides, lipids, phospholipids, sugars, polysaccharides, and combinations thereof. Specific examples of analyte can include proteins, metabolites, allergens, hormones, toxins, RNA (e.g., mRNA, total RNA, tRNA, etc.), DNA (e.g., plasmid DNA, plant DNA, etc.), tagged proteins, antibodies, antigens, and combinations thereof, but are not limited to these.

The terms “polypeptide”, “peptide”, and “protein” are used interchangeably herein to designate a linear series of amino acid residues connected one to the other by peptide bonds, which includes proteins, polypeptides, oligopeptides, peptides, and fragments thereof. The protein can be made up of naturally occurring amino acids and/or synthetic (e.g. modified or non-naturally occurring) amino acids. Thus “amino acid”, or “peptide residue”, as used herein means both naturally occurring and synthetic amino acids. The terms “polypeptide”, “peptide”, and “protein” includes fusion proteins, including, but not limited to, fusion proteins with a heterologous amino acid sequence, fusions with heterologous and homologous leader sequences; immunologically tagged proteins; fusion proteins with detectable fusion partners, e.g., fusion proteins including as a fusion partner a fluorescent protein, β-galactosidase, luciferase, etc.; and the like.

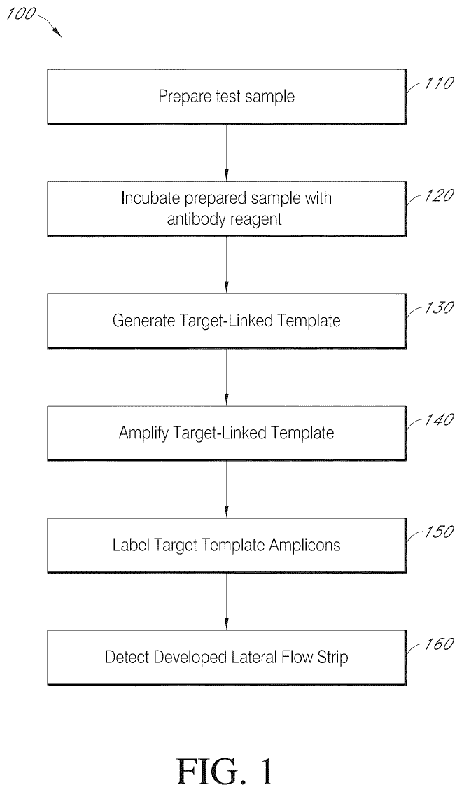

FIG. 1 illustrates generally a work flow process for performing a protein detection assay in accordance with various embodiments of the present disclosure. At step 110 of the workflow, a target sample, for example, a urine sample is prepared for analysis before an aliquot of the prepared sample is introduced to a sample compartment of a lateral flow device. For point of care analysis of IP-10, for which increasing levels can be an indication of kidney rejection, urine can be a target sample. For example, for point of care testing, it is easy for an end user to provide a urine sample with no intervention of medical personnel required. Additionally, urine is often a sample of choice for analyses related to various kidney diseases. As will be described in more detail herein, given the complex nature of urine as a sample, the present inventors have found that simple dilution of a urine sample was not sufficient to mitigate interferants in a urine sample from causing untoward effects on an assay, such as, for example, non-specific background color development on a lateral flow substrate of a lateral flow device.

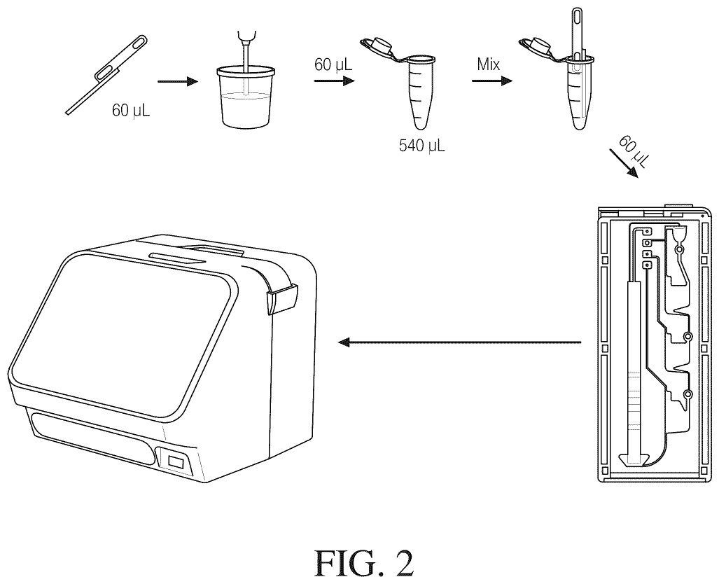

In that regard, as illustrated schematically in FIG. 2, an end user can use a disposable volumetric pipette, to take a measured aliquot of a urine sample from a urine sample container and add it sample holding device, such as, for example a test tube, a vial or microfuge tube. As depicted in FIG. 2, the undiluted urine sample can be added to the sample container that contains measured volume of a sample diluent. As depicted in FIG. 2, the dilution represents a 1:10 dilution of a urine sample. The present inventors discovered that a sample diluent containing DMSO was effective in mitigating untoward effects of interferants in urine on a PLA-LF assay. In some formulations of a sample diluent, a 0.5% (v/v) to 5% (v/v) DMSO sample diluent solution can be prepared using ultra high purity DNase/RNase free distilled water. In an alternative formulations of a sample diluent, a 0.5% (v/v) to 5% (v/v) DMSO sample diluent solution can be prepared in a 25 mM-50 mM Tris buffer prepared using ultra high purity DNase/RNase free distilled water. It should be noted that the present inventors discovered that phosphate-based buffers, such as phosphate buffered saline (PBS), that were used to prepare a DMSO-based sample diluent produced untoward non-specific background color development on a lateral flow substrate. For performing step 110 of FIG. 1, and with respect to the work flow process depicted in FIG. 2, according to the present disclosure, consumables required for preparing a urine sample, for example, disposable volumetric pipettes, diluent and disposable lateral flow device, can be provided in a kit.

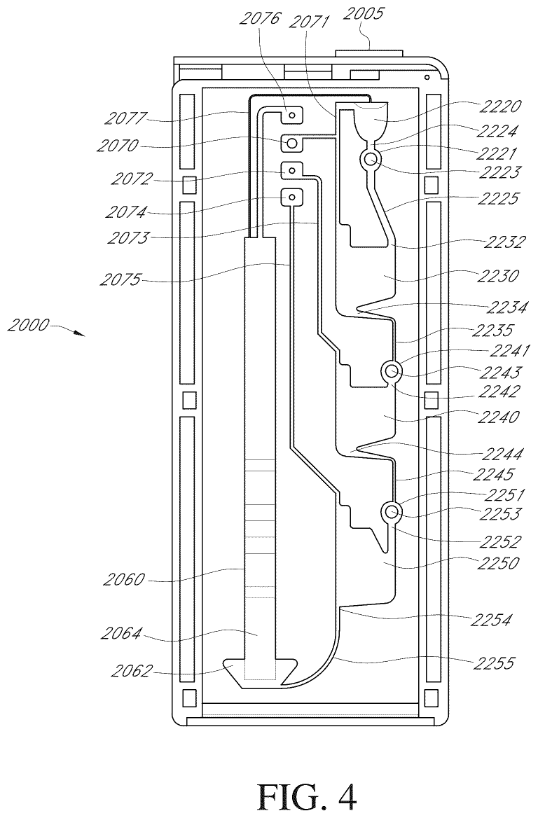

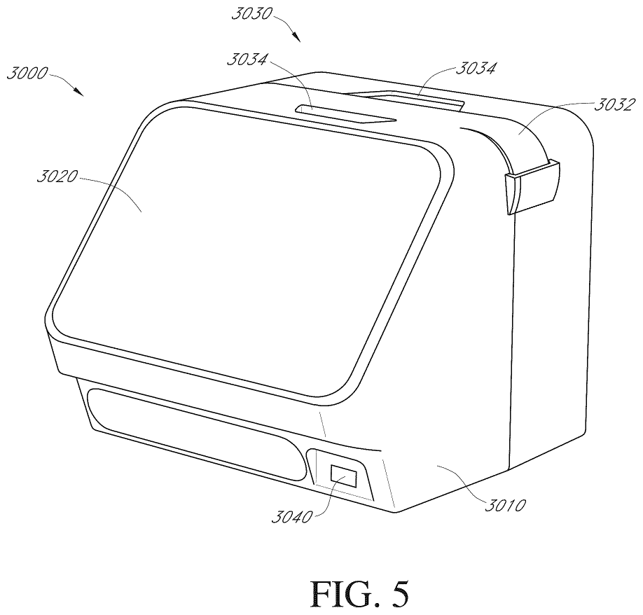

At step 120 of the workflow, a sample in a first processing chamber can be incubated with an antibody reagent. A lateral flow device, such as lateral flow device 2000 of FIG. 4; can be inserted into an instrument, such as instrument 3000 of FIG. 5; also as depicted in FIG. 2. As illustrated in FIGS. 2, 60 μL of the sample solution prepared as described for step 110 of FIG. 1 can be introduced into a lateral flow device. As will be described in more detail herein, once the sample is introduced into a lateral flow device, it can flow into a first processing chamber, where it can be incubated with an antibody conjugate reagent. For example, after the sample has been introduced to a lateral flow initiated, the sample can be incubated in a first chamber for at least 15 minutes.

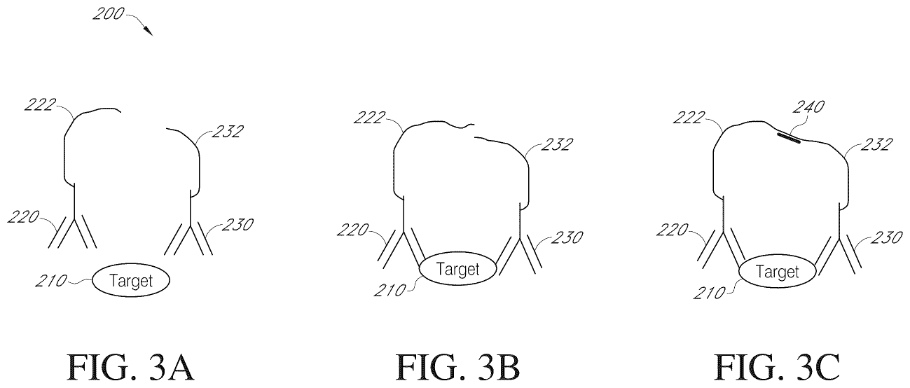

At step 130 of the workflow, an IP-10-linked template nucleic acid can be generated. FIG. 3A through FIG. 3C are schematic diagrams that generally illustrate of stages of a proximity assay in accordance with various embodiments the present disclosure. As described for step 130 of FIG. 1, target-linked template generation provides template nucleic acid, which is then amplified in a subsequent amplification step, for example, as described for step 120 of FIG. 1. FIG. 3A through FIG. 3C schematically illustrate target-linked template generation for PLA in accordance with embodiments of the present disclosure. FIG. 3A shows various stages and components of PLA reaction 200 for generating a single strand of template nucleic acid from a target analyte 210 from which a double-stranded template nucleic acid can be generated for subsequent amplification. The components of the PLA reaction 200 depicted include a first antibody or antibody fragment 220 specific to a first epitope of a target analyte protein 210 and a second antibody or antibody fragment 230 specific to a second epitope of the target analyte protein 210. An antibody or antibody fragment can be substituted with a DNA aptamer or other binding moiety with specificity for the respective target epitope. A first oligonucleotide 222 is bound at its 5′-end to first antibody 220 with its 3′-end free to form a first antibody conjugate. A second oligonucleotide 232 is attached at its 3′-end to second antibody 230 with its 5′-end free to form a second antibody conjugate. In FIG. 3B, first and second antibodies 220, 230 bind to target analyte 210 due to their affinity for the respective first and second epitopes of target analyte 210. In FIG. 3C, with the first and second antibodies 220, 230 bound to analyte 210, a splint oligonucleotide 240, along with a ligase reagent, is used to ligate the end portions of the first and second oligonucleotides 222, 232. The result is a proximity oligonucleotide product that comprises the joined first oligonucleotide 222, the second oligonucleotide 232, and the third, splint oligonucleotide 240. The resulting proximity oligonucleotide product thus provides a single strand of the template nucleic acid (the joined strands 222, 232) for generating a double-stranded template to be used in an amplification reaction. As will be described in more detail herein, a PLA reagent can be formulated to include all necessary components for performing ligation.

At step 140, after target-linked template generation occurs, template nucleic acid amplification is performed on any of the template nucleic acid product that may be generated at 130. Thus, at step 140, assuming the initial presence of the target protein and subsequent template nucleic acid generation at 130, amplification product (e.g., copy amplicons of the template nucleic acid) is generated at 140. Amplification can occur, for example, via polymerase chain reaction (PCR) or rolling circle amplification. Thermal cycling or isothermal amplification can be used.

After amplification of template nucleic acid has occurred to produce an amplification product, amplicon labeling occurs at step 150. Here, the amplicons generated via amplification of template nucleic acid are individually labeled to produce a labeled product. For example, detection probes can be introduced to the amplification product to bind to the amplicons. Labeling may occur via probes attached to colored particles, such as beads, or via probes carrying fluorescent dye, for example. But such are not limited and additional detection probe embodiments for performing labeling of amplicon are envisioned as within the scope of the present disclosure.

Detection probe as used herein refers to a conjugate used to label nucleic acid product (amplicons) generated during an amplification reaction, and includes fluorescent dyes specific for duplex nucleic acid, dye-modified oligonucleotides, such as fluorescently-dye modified oligonucleotides, oligonucleotide-conjugated quantum dots, or oligonucleotide-conjugated solid phase elements such as a polystyrene, latex or paramagnetic particles or microspheres. As used herein, beads, particles and microspheres can be used interchangeably. Suitable detection probes include but are not limited to a range of dyes including visible dyes and fluorescent dyes specific for duplex nucleic acid, fluorescently modified oligonucleotides, or oligonucleotide-conjugated dyed microparticles or colloidal gold. Detection of amplicon involves the “detection oligonucleotide” of the detection probe that is complementary to or otherwise able to bind specifically to the amplicon to be detected. Conjugation of a detection oligonucleotide to a microparticle can occur by use of streptavidin coated particles and biotinylated oligonucleotides, or by carbodiimide chemistry whereby carboxylated particles can be activated in the presence of carbodiimide and react specifically with primary amines present on the detection oligonucleotide. Conjugation of the detection oligonucleotide to the detectable moiety can occur internally or at the 5′ end or the 3′ end. Detection oligonucleotides can be attached directly to a detection label (e.g., the portion of the detection probe that provide for a detectable emission such as fluorescence, color, etc.), or more, for example, through a spacer moiety such as ethylene glycol or polynucleotides.

Once the amplicons are bound with the labeled probes, labeled amplicons are created and can be detected at step 160 of FIG. 1 In an embodiment, the detection occurs through the a capturing moiety configured to bind to and selectively capture the labeled amplicons. Such capturing moiety can be provided on a lateral flow substrate, which can be a solid or semi-solid phase placed in fluid communication with the labeled product. For example, a lateral flow substrate can be an absorbent porous matrix material, for example, such as nitrocellulose, cellulose, polyethersulfone, polyvinylidine fluoride, nylon, charge[modified nylon and polytetrafluoroethylene, a gel, various surfaces of a chamber or channel, as well as a particulate bed. As will be disclosed in more detail herein, a lateral flow substrate can be fabricated in various formats such as a lateral flow strip or a corrugated structure, etc. Depending on the labeling technique employed, detection of labeled amplicons can be via observation of color development (colorimetric detection) or sensing fluorescence by an optical sensor, or a combination of both.

FIG. 4 illustrates generally lateral flow device 2000 of the present disclosure. The fluidic network of lateral flow device 2000 includes sample chamber 2220 which is in fluid communication with sample port 2005. In FIG. 4, proximal to sample chamber outlet 2224 is first reagent recess 2221 containing first reagent pellet 2223. As a sample passes through first reagent recess 2221, mixing of the sample with dried or lyophilized reagents contained therein is promoted. As previously described herein, effective mixing provides a uniform solution and thereby a uniform reaction mixture. As will be described in more detail herein, first reagent pellet 2223 can include a variety of reagents related to PLA analysis, such as reagents for sample pretreatment prior to processing a sample for PLA analysis or such as antibody conjugate reagents. First fluidic channel 2225 is in fluid communication with first processing chamber 2230, which includes first processing chamber inlet 2232 and first processing chamber outlet 2234. First processing chamber 2230 can be utilized to as for processing a sample for pretreatment or to incubate a sample with an antibody conjugate reagent prior to preparation of protein-target-linked template generation and amplification as described for steps 110 and 120 of FIG. 1.

Second fluidic channel 2235 is proximal to first processing chamber outlet 2234 and is in fluid communication with second processing chamber 2240. Proximal to second sample chamber inlet 2242 is second reagent recess 2241 containing second reagent pellet 2243. As a sample passes through second reagent recess 2241, mixing of the sample with dried or lyophilized reagents contained therein provides a uniform solution and thereby a uniform reaction mixture. As will be described in more detail herein, second processing chamber 2240 can be utilized for a variety of PLA processing steps. As such, second reagent recess 2241 can have a second reagent pellet 2243 varying on composition depending on what processing step is accommodated in second processing chamber 2240. For example, second reagent pellet 2243 can include reagents related to various processing steps of PLA analysis, such as antibody conjugate reagent, or ligation reagents or a combination of ligation and PCR reagents. Accordingly, processing in second processing chamber 2240 can include incubation of the sample with antibody conjugate reagents, incubation with ligation reagents, or incubation with a combination of ligation and PCR reagents.

Third fluidic channel 2245 is proximal to second processing chamber outlet 2244 and is in fluid communication with third processing chamber 2250. Proximal to third sample chamber inlet 2252 is third reagent recess 2251 containing third reagent pellet 2253. As a sample passes through third reagent recess 2251, mixing of the sample with dried or lyophilized reagents contained therein is provides a uniform solution and thereby a uniform reaction mixture. As will be described in more detail herein, third processing chamber 2250 can be utilized for a variety of PLA processing steps. As such, third reagent recess 2251 can have a third reagent pellet 2253 varying on composition depending on what processing step is accommodated in third processing chamber 2250. For example, third reagent pellet 2253 can include reagents related to various processing steps of PLA analysis, such PCR reagents, or a combination of ligation and PCR reagents or detection probe labeling reagents. Accordingly, processing in third processing chamber 2240 can include incubation of the sample with PCR reagents. incubation with a combination of ligation and PCR reagents or incubation with detection probe labeling reagents.

As previously described herein for lateral flow device 2000, dried or lyophilized sample pretreatment reagents can include reagents for pH or ionic strength adjustment, or depending on the nature of the sample, a lysing reagent. Dried or lyophilized antibody conjugate reagents contain two antibody conjugates, each coupled to complementary oligonucleotide strands and each directed to a different epitope of a target protein as described for FIG. 3A through FIG. 3C. Accordingly, a dried or lyophilized antibody conjugate reagent pellet can contain buffering agents, salts, antibody conjugates, as well as stabilizing excipients, such as polyols, surfactants and antioxidants. Dried or lyophilized ligation reagents can include buffering agents, salts, ATP and a ligase enzyme, while dried or lyophilized reagents containing both ligation and PCR reagents can contain buffering agents, salts, ATP and a ligase enzyme, as well as all components necessary for the amplification reaction, for example such as buffering agents, salts, dNTPs, rNTPs, forward and reverse primers, as well as a heat stable polymerase. Additionally, a positive control such as a virus, bacteria or nucleic acid can be included in a dried or lyophilized formulation including amplification reagents.

Various processes conducted in various chambers of lateral flow device 2000 to carry out a PLA or PEA assay or both as described for method 100 of FIG. 1 can vary for various embodiments of fluidic device 2500 of FIG. 4 utilizing lateral flow device 2000.

By way of a first non-limiting example, first reagent pellet 2023 contained in first recess 2221 of FIG. 4 can include antibody conjugate reagents for forming a protein-antibody conjugate complex with a target protein if present in the sample, and incubation of the sample with the antibody conjugate reagents can be performed in first processing chamber 2230. Second reagent pellet 2243 formed in second reagent recess 2241 can contain ligation reagents for forming target-linked template nucleic acids, and incubation of the sample with the ligation reagents can be performed in second processing chamber 2240. Third reagent pellet 2253 contained in third reagent recess 2251 can contain PCR reagents for forming template nucleic acid amplicons, and amplification can be performed in third processing chamber 2250. As will be described in more detail herein, template nucleic acid amplicons formed in processing chamber 2250 can be labeled with detection probes in detection chamber 2060.

In a second non-limiting example, first reagent pellet 2023 contained in first recess 2221 of FIG. 4 can include reagents for sample pretreatment prior to processing a sample for PLA analysis, for example, for reagents for pH or ionic strength adjustment, or depending on the nature of the sample, a lysing reagent. Preprocessing of a sample can be performed in first processing chamber 2230. Second reagent pellet 2243 formed in second reagent recess 2241 can contain antibody conjugate reagents for forming a protein-antibody conjugate complex with a target protein if present in the sample, and incubation of the sample with the antibody conjugate reagents can be performed in second processing chamber 2240. Third reagent pellet 2253 contained in third reagent recess 2251 can contain ligation reagents for template nucleic acid formation, as well as PCR reagents for amplification, and processing to form template nucleic acids and template nucleic acid amplicons can be performed in processing chamber 2250. Template nucleic acid amplicons formed in processing chamber 2250 can be labeled with detection probes in detection chamber 2060.

In a third non-limiting example, first reagent pellet 2023 contained in first recess 2221 of FIG. 4 can include antibody conjugate reagents for forming a protein-antibody conjugate complex with a target protein if present in the sample, and incubation of the sample with the antibody conjugate reagents can be performed in first processing chamber 2230. Second reagent pellet 2243 contained in second reagent recess 2241 can contain ligation reagents for template nucleic acid formation, as well as PCR reagents for amplification, and processing to form template nucleic acids and template nucleic acid amplicons can be performed in processing chamber 2240. Third reagent pellet 2253 contained in third reagent recess 2251 can contain detection probe labeling reagents for labeling template nucleic acid amplicons, and labeling can be performed in processing chamber 2250. A sample with detection labeled amplicons formed in processing chamber can flow into fluid compartment 2062 of detection chamber 2060, and develop on a lateral flow substrate, such as lateral flow strip 2064.

In a forth non-limiting example, first reagent pellet 2023 contained in first recess 2221 of FIG. 4 can include antibody conjugate reagents for forming a protein-antibody conjugate complex with a target protein if present in the sample, and incubation of the sample with the antibody conjugate reagents can be performed in either first processing chamber 2230 or second processing chamber 2240. Third reagent pellet 2253 contained in third reagent recess 2251 can contain ligation reagents for template nucleic acid formation, as well as PCR reagents for amplification, and processing to form template nucleic acids and template nucleic acid amplicons can be performed in processing chamber 2250. Template nucleic acid amplicons formed in processing chamber 2250 can be labeled with detection probes in detection chamber 2060.

Fluid compartment 2062 of FIG. 4 can contain lyophilized detection reagents including detection probes. As previously described herein, a detection probe is a conjugate of a detection oligonucleotide that is complementary to a template nucleic acid or to a control and a detectable microsphere providing for visual and optical detection. Suitable detection probes include oligonucleotide conjugated to a detectable solid support, such as a polystyrene microspheres, latex beads or paramagnetic beads. Such detectable solid-supports can incorporate a range of detectable moieties including visible dyes, fluorescent dyes, quantum dots or colloidal gold. Accordingly, a dried or lyophilized detection reagent pellet can contain buffering agents, salts, and detection probes.

Additionally, fluid compartment 2062 of FIG. 4 provides a space of sufficient capacity to accommodate substantially all or the entire volume of fluid in the detection chamber at a height enabling flow up a lateral substrate, such as lateral flow strip 2064 of FIG. 4, without flooding and ensuring correct capillary migration up the lateral flow detection strip to ensure effective detection. In that regard, for efficient labeling to occur, sample entering fluid compartment 2062 from third fluidic channel 2045 can mix with lyophilized detection reagents. Fluid compartment 2062 is configured to provide effective mixing and dispersion of particles to facilitate a uniform reaction mixture, as well as increasing the uniformity of particle migration on lateral flow strip 2064. Fluid compartment 2062 of FIG. 4 can be especially advantageous for low volume assays, such as assays with a volume less than 200 μL, or more specifically less than between about 40 μL to about 100 μL in volume.

As previously described herein regarding a lateral flow substrate, such as lateral flow strip 2064 of FIG. 4, capture probes can be contained in or on a lateral flow substrate, which can be a porous matrix material, for example, such as nitrocellulose, cellulose, polyethersulfone, polyvinylidine fluoride, nylon, charge[modified nylon and polytetrafluoroethylene. Capture probes can be concentrated at a location defining a test line extending transverse to the capillary flow direction through a lateral flow substrate. Capture probes can patterned on a lateral flow substrate in other shapes, for example dots, a plus sign, a minus sign, etc. Capture probes are configured to capture a detection oligonucleotide conjugated to a detection probe. When a sufficient level of labeled target template amplicons are captured, the concentration of detection probes along a test line or other pattern becomes visually and optically detectable. As such, sufficient concentration of detection probe immobilized by patterned capture probe on a lateral flow substrate provides an indication of presence of a detectable concentration of a target-linked template nucleic acid or control.

For lateral flow device 2000 as depicted in FIG. 4, a sample introduced into sample port 2005 can flow to sample chamber 2220 and then to first processing chamber 2230, as first processing chamber 2230 is vented to an expansion volume (not shown) via vent channel 2071, which is in fluid communication with unsealed vent pocket 2070. Fluid movement from first processing chamber 2230 to second processing chamber 2240 is accomplished by actuating the opening of vent pocket 2072 connected to second processing chamber 2240 via vent channel 2073. When fluid enters first processing chamber 2230, vent pocket 2072, connected to second processing chamber 2240, is sealed, and thus fluid will not pass through second fluidic channel 2235 connecting the two chambers. The rupture of a seal of vent pocket 2072 allows vent pocket 2072 and thus second processing chamber 2240 to be in fluid communicate with unsealed vent pocket 2070 and thereby with an expansion volume (not shown). For lateral flow device 2000, fluid movement from second processing chamber 2240 to third processing chamber 2250 is accomplished by actuating the opening of vent pocket 2074 connected to third processing chamber 2250 via vent channel 2075. Rupture of a seal of vent pocket 2074 allows vent pocket 2074 and thus processing chamber 2250 to be in fluid communicate with unsealed vent pocket 2070 and thereby with an expansion volume (not shown). Fluid movement from third processing chamber 2250 to detection chamber 2060 is accomplished by actuating the opening of vent pocket 2076 connected to detection chamber 2060 via vent channel 2077. Rupture of a seal of vent pocket 2076 allows vent pocket 2076 and thus detection chamber 2060 to be in fluid communicate with unsealed vent pocket 2070 and thereby with an expansion volume (not shown). As such, this method of fluid movement provides for containment of sample fluids within a hermetically sealed expansion volume, thereby containing biohazard associated with various biological samples within the fluidic device.

FIG. 5 is a front perspective view that illustrates generally instrument 3000 for receiving a lateral flow device of the present disclosure, such as lateral flow device 2000 of FIG. 4, and carrying out an analysis. Instrument 3000 of FIG. 5 can include a housing with base portion 3010, front face comprising a display 3020, and lateral flow device insertion portion 3030. Display 3020 can be an LCD display in one embodiment and can communicate information such as workflow (assay) protocols and status to a user. Additionally, display 3020 can be a touchscreen display and configured to accept user input, for example, to provide control parameters for workflows to be carried out. Instrument 3000 can also include one or more external connector interfaces 3040 that can be configured to connect to various transmission conduits, such as electrical transmission conducts so as to transmit power from an external power source to the instrument 3000, data transmission conduits to transmit data, and/or combined conduits to transmit power and data to various devices, such as peripheral devices including printers, monitors, etc., and/or to networked computing device and/or telecommunications devices.

Instrument 3000 can include an assembly for receiving a lateral flow device, such as device receiving assembly 3030 of FIG. 5. Device receiving assembly can include device receiving assembly cover 3032, that when lifted, allows a lateral flow device to be inserted into instrument aperture 3034. Aperture 3034 is configured to receive a lateral flow device, such as lateral flow 2000 of FIG. 4, so that a lateral flow device is engaged with the interior of instrument 3000 so as to provide registration of a lateral flow device with various components located in the interior of instrument 3000 essential for automated processing of a lateral flow device. Aperture 3034 can have dimensions that provide a tight tolerance with the lateral flow device to ensure proper insertion and a secure fit when inserted into the instrument 3000.

Insertion of a lateral flow device into the instrument 3000 through aperture 3034 places the lateral flow device in an orientation for promoting gravity-driven flow. Additionally, once a lateral flow device is inserted into instrument 3000, a lateral flow device is positioned to permit various portions of the instrument 3000 to interact with the lateral flow device. For example, in an inserted position in the instrument 3000, the lateral flow device is placed in a desired positioning for various sensors to obtain data from the lateral flow device, such as for thermal control, fluidics flow handling, monitoring of portions of an overall workflow, and detection of test results. In addition, the lateral flow device is placed in electrical communication with a printed circuit assembly of the instrument so as to provide the transmission of current to the various addressable thermal energy generation elements and other control elements of the circuit board component of the lateral flow device.

### Overview of Analysis of Interferon Gamma-Induced Protein 10 (IP-10)

As previously described herein, interferon gamma-induced protein 10, IP-10, can be used to monitor kidney transplant rejection. For point-of-care (POC) testing, a detection level of about 10-15 pg/ml in urine is required. A differentiating feature of incorporation of PLA into a microdevice format, such as a lateral flow (LF) platform is that there can be substantially more protein in the PLA assay format than would be included in various analyses in which the analyte is nucleic acid-based. For example, in the PLA format, an antibody reagent is used, which can require the use of bulking protein agents to provide stabilization. As such, an antibody reagent of a PLA assay can add substantially more protein than a nucleic acid-based assay, for which a polymerase may be the only source of protein in an assay.

Addressing the issue of protein precipitation for protein analysis in a lateral flow format presented a formidable challenge that the present inventors addressed. By way of perspective, in a macro format, for example, in test tube, microfuge tube or PCR plate format, in a fully-equipped laboratory with trained personnel, precipitation during an analysis workflow can be readily addressed through, for example, filtration or centrifugation. In contrast, such precipitation in a sample-in-answer-out microdevice format can result in inconsistent results, and moreover may result in failure.

The present inventors have observed that protein precipitates can cause fluid to be trapped at the bottom of a lateral flow substrate, such as a lateral flow strip, can cause uneven signal bands, and can cause channel blocking, thereby rendering a lateral flow device unusable. Protein precipitation was observed upon heat treatment to inactivate ligase. Given the complex nature of the mixture that included a polymerase, enzymatic deactivation of ligase using a protease was contraindicated. Initially, formulations of a PLA reagent that utilized AmpliTaq™ fast DNA polymerase produced protein precipitation. However, surprisingly, in testing PLA reagent formulations using the Lyo-ready Phire™ hot start II DNA polymerase (Thermo Fisher part #EP1980SMP100), no precipitation occurred.

Additionally, given the complex nature of urine as a sample, and given that that interferants in urine samples have been demonstrated by the present inventors to be problematic in a PLA-LF platform, an effective approach for the mitigation of interferants in urine samples is desirable for a PLA-LF platform targeted to POC testing. As will be described herein subsequently, the use of a diluent formulated using dimethyl sulfoxide (DMSO) was effective in the mitigation of interferants in urine samples.

As such, the present inventors have discovered how to effectively treat urine samples to reduce background interference for detection using a PLA-LF platform, as well as to develop reagents capable of being formed readily into a stable pellet, each reagent pellet formulated to substantially reduce precipitate formation. In a PLA reagent formulation including a polymerase, the present inventors discovered a polymerase that surprisingly reduces precipitate formation, which contributed to the effectiveness of a PLA reagent of the present disclosure.

### Method For Analysis of Interferon Gamma-Induced Protein 10 (IP-10)

### Preparation of Reagents:

A formulation of a urine sample diluent was prepared using an aqueous dilution of between 0.5% (v/v) to 5% (v/v) dimethyl sulfoxide (DMSO). The present inventors found that dilution of urine with a diluent containing DMSO in that concentration range was important for substantially reducing or eliminating untoward non-specific background color development on a lateral flow strip. In some formulations of a sample diluent, a 0.5% (v/v) to 5% (v/v) DMSO sample diluent solution can be prepared using ultra high purity DNase/RNase free distilled water. In an alternative formulations of a sample diluent, a 0.5% (v/v) to 5% (v/v) DMSO sample diluent solution can be prepared in a 25 mM-50 mM Tris buffer prepared using ultra high purity DNase/RNase free distilled water. It should be noted that the present inventors discovered that phosphate-based buffers, such as phosphate buffered saline (PBS), that were used to prepare a DMSO-based sample diluent produced untoward non-specific background color development on a lateral flow strip.

Antibody conjugates for IP-10 from ProQuantum Immunoassay Kit (Thermo Fisher catalog #A35578) were used. A bulk formulation of an antibody conjugate solution was prepared using ratio (v/v) one part of each antibody conjugate A and antibody conjugate B diluted with 23 parts of a polysaccharide diluent and 32 parts of phosphate buffered saline (PBS) prepared using ultra high purity DNase/RNase free distilled water. The polysaccharide diluent was a mixture of 1M sucrose and 1M trehalose prepared in phosphate buffered saline (PBS) prepared using ultra high purity DNase/RNase free distilled water. An antibody conjugate reagent pellet formed in each cassette was formed using 5 μL of a bulk antibody conjugate solution so formulated.

In order to utilize a single processing compartment of a lateral flow test device for ligation and PCR, such as described for chamber 2250 of cassette device 2000 of FIG. 4, a formulation of PLA reagent that combined ligase/amplification/positive control reagent was prepared. A bulk formulation of a PLA reagents was prepared using components from the ProQuantum Immunoassay Kit that included for the formation of a template nucleic acid a final concentration of 30 nM for the splint, as well as 0.5-1 U/pellet of SplintR® ligase.

For performing the amplification of template nucleic acid formed in the ligation step, the bulk formulation of the PLA reagent also included 100 μM of the forward and reverse IP- 10 primers, as well as 8 U/pellet of Lyo-ready Phire™ hot start II DNA polymerase (Thermo Fisher part #EP1980SMP100). The Lyo-ready Phire™ hot start II DNA polymerase was very stable in the buffer diluent used for formulating the PLA reagent, and is formulated to be readily prepared as a dried or lyophilized reagent. In contrast, many commercially-available polymerase preparations; particularly in liquid form, can have high concentrations of glycerol in the preparation. The presence of high concentrations of glycerol presented problems with respect to lyophilization of a PLA reagent. Moreover, as previously described here, the use of the Lyo-ready Phire™ hot start II DNA polymerase prevented untoward protein precipitation, thereby enabling consistent results.

Additionally, the bulk formulation included a positive control was a 141 nucleotide (nt) MS 2 ultramer in final concentration of 2 pM, and also included the associated primers; the forward primer in a final concentration of 0.4 μM and the reverse primer in a final concentration of 0.2 μM. The bulk PLA reagent formulation included concentration of 200 μM for each deoxynucleotide triphosphate dNTP (Thermo Fisher part #R 01820). A 0.45M tris buffer containing additives essential for PCR was used as the diluent for the bulk PLA reagent formulation. The 0.45M tris buffer was prepared using ultra high purity DNase/RNase free distilled water and also included 0.23M sucrose, 0.57M trehalose, 187 mM ammonium sulfate, 0.7M potassium chloride, 0.56% (v/v) Triton™ X-100, 56 mM magnesium sulfate, 0.64mg/ml of bovine serum albumin (BSA) and 0.15M dithiothreitol (DTT). A PLA reagent pellet formed in each cassette was formed using 6 μL of a bulk PLA reagent solution as described herein.

### Automated IP-10 PLA Analysis Using a Lateral Flow Device Platform:

For performing an automated analysis of IP-10, lateral flow devices, such as described for lateral flow device 2200 of FIG. 4 were used. For each test sample run, a lateral flow device was placed in an, such as described for FIG. 5.

Test samples stock solutions of 10 pg/ml and 100 pg/ml of IP-10 synthetic analyte (SA) provided with the ProQuantum Immunoassay kit were prepared in urine. For the purpose of testing, the sample stock solutions were then diluted as previously described herein with either an aqueous DMSO sample diluent solution or a DMSO sample diluent solution in a 25-50 mM Tris buffer. As such, the test samples were analyzed using the 1 pg/ml and 10 pg/ml IP-10 solutions diluted with a DMSO sample diluent solution. Each test sample was run on a separate lateral flow device. A sample of assay buffer was used to as a control for background. A lateral flow device, such as lateral flow device 2000 of FIG. 4, was inserted into an instrument, such as instrument 3000 of FIG. 5. 60 μL of the sample solution as prepared as described was added to a sample chamber, such as sample chamber 2220 of FIG. 4. As the sample flowed from the sample chamber to a first processing chamber, such as chamber 2230 or chamber 2240 of FIG. 4, it mixed with an antibody conjugate pellet formed in a reagent recess, such as reagent recess 2221 of FIG. 4. The antibody conjugate pellet formed in the reagent recess was prepared as previously described herein. When the analysis was initiated, the sample mixed with the antibody conjugate reagent was allowed to incubate in the first chamber for 15 minutes. Upon automated actuation to open a first vent, the sample/antibody conjugate mixture passed into a second sample processing chamber, such as chamber 2250 of FIG. 4. As the sample flowed from the first processing chamber to the second processing chamber, it mixed with a PLA reagent pellet formed in a second reagent recess, such as reagent recess 2251 of FIG. 4. The PLA reagent pellet was prepared as previously described herein. In order to form a template nucleic acid sample during a ligation step, the sample was allowed to incubate in the second chamber for 10 minutes at 20 °C, followed by ligase inactivation at 98 °C for 2 minutes. After the inactivation of ligase, PCR cycling was automatically initiated, where each cycle included 98 °C for 15 seconds and 54 °C for 25 second. A total of 33 cycles of PCR was performed in the processing chamber, which took about 35 minutes.

Upon completion of amplification, the reaction mixture reconstituted lyophilized detection particles as it flowed into the detection chamber. The lateral flow strip was allowed to develop for 5 minutes prior to automated analysis of the lateral flow strip by the instrument. Capture lines were detected on the lateral flow membrane for the 1 pg/ml and 10 pg/ml test samples on their respective lateral flow devices. From the bottom of the device, the first capture line was for a capture probe complementary to the amplification product of IP-10; and the second capture line was for a oligonucleotide complementary to the amplification product of the MS2 control. For the 0 pg/ml background control, only a single capture line for the MS 2 control was observed.

Accordingly, automated detection of IP-10 an order of magnitude below the target range of 10-15 pg/ml was observed.

As such, according to the present disclosure, in a first example, a method for automated detection of IP-10 in a sample comprises processing the sample with an aqueous solution containing dimethyl sulfoxide (DMSO) in a range of between about 0.5% (v/v) to about 2.0% (v/v); introducing the sample into a sample chamber of a cassette, the cassette including a plurality of processing chambers; opening a first vent to allow the sample mixed with an antibody conjugate sample in a first processing chamber to flow to a thermally controlled second processing chamber configured to form a detectable amount of an IP-10 template nucleic acid amplicon sample in response to the presence of an IP-10-linked template nucleic acid; opening a second vent to allow the IP-10 template nucleic acid amplicon sample from the second processing chamber to come in contact with a labeling reagent, thereby generating a labeled template nucleic acid amplicon sample in response to the presence of IP-10 template nucleic acid amplicon; and contacting a lateral flow substrate with the labeled template nucleic acid amplicon sample; said lateral flow substrate configured to generate a signal at a capture line on the lateral flow substrate in response to the presence of IP-10 in the sample.

A second example includes the method of the first example, and further specifies that processing the sample with an aqueous solution of DMOS in a range between about Example 0.5% (v/v) to about Example 2.0% (v/v) comprises diluting the sample.

A third example includes the method of the second example, and further specifies that diluting the sample with an aqueous solution of DMOS in a range between about Example 0.5% (v/v) to about Example 2.0% (v/v) comprises a volumetric dilution of 1:Example 10.

A fourth example includes the method of any one of the first through third examples, and further specifies that the aqueous solution of DMSO in a range between about 0.5% (v/v) to about 2.0% (v/v) is a buffered aqueous solution.

A fifth example includes the method of the fourth example and further specifies that the aqueous solution of DMSO in a range between about 0.5% (v/v) to about 2.0% (v/v) is buffered using a 25-50 mM Tris buffer.

A sixth example includes the method of any one of the first through fifth examples, and further specifies that the signal at the capture line on the lateral flow substrate indicative of IP-10 is present at a detection limit of 1 pg/mL of IP-10 in a urine sample.

A seventh example includes the method of any one of the first through sixth examples, and further specifies that the sample is from a subject having or suspected of having a disease.

An eighth example includes the method of any one of the first through seventh examples, and further specifies that the disease is a renal disease.

A ninth example includes the method of any one of the first through the eighth examples, and further specifies that the substrate is a lateral flow strip.

A tenth example includes the method of the ninth example, and further specifies that the lateral flow strip comprises a porous matrix.

In an eleventh example, a disposable cassette for detecting IP-10 comprises a sample receiving zone configured to receive and retain a sample; an antibody reagent pellet in a first reagent recess proximal to a sample chamber outlet of the sample receiving zone; a first chamber in fluid communication with the sample receiving zone and the first reagent recess; a second chamber in controllable fluid communication with the first chamber, said second chamber in fluid communication with a second reagent recess proximal to an inlet of the first chamber, said second reagent recess containing a PLA reagent pellet, which includes a ligase for the preparation of a proximity oligonucleotide product; and a polymerase for amplification of an IP-10 target-linked oligonucleotide; the disposable cassette further including a detection chamber for detection of said IP-10 target-linked oligonucleotide or amplicons thereof, said detection chamber including a lateral flow substrate patterned with a binding moiety specific for at least a portion of said IP-10 target-linked oligonucleotide or amplicons thereof.

A twelfth example includes the disposable cassette of the eleventh example, and further specifies that said detection chamber further comprises a fluid compartment containing detection particles for labeling amplicons of the IP-10 target-linked oligonucleotide.

A thirteenth example includes the disposable cassette of either of the eleventh or twelfth examples, and further specifies that said detection chamber is downstream of and in fluidic connection with the first chamber.

A fourteen example includes the disposable cassette of any one of the eleventh through thirteenth examples, and comprises a plurality of chambers including the first chamber, the second chamber, and the detection chamber.

A fifteenth example includes the disposable cassette of any one of the eleventh through fourteenth examples, and further includes a plurality of vents in connection with said plurality of chambers.

A sixteenth example includes the disposable cassette of any one of the eleventh through fifteenth examples, and further includes a fluidic network configured to control and regulate the flow of the sample.

A seventeenth example includes the disposable cassette of the sixteenth examples, and further specifies that the fluidic network comprises a plurality of fluidic channels.

An eighteenth example includes the disposable cassette of any one of the first through the seventeenth examples, and further specifies that the polymerase is a hot start DNA polymerase.

A nineteenth example includes the disposable cassette of the eighteenth example, and further specifies that the hot start polymerase is Lyo-ready Phire™ hot start II DNA polymerase.

In a twentieth example, a kit for performing point of care testing for IP-10 in a sample, the kit comprising a lateral flow device configured for assaying said sample, said cassette including a first processing chamber configured for processing said sample; a second processing chamber configured for generating an IP-10 target-linked oligonucleotide or amplicons thereof; and a detection chamber for detection of said IP-10 target-linked oligonucleotide or amplicons thereof; the kit further including one or more disposable volumetric pipettes; and a buffer containing dimethyl sulfoxide (DMSO) in a range between about 0.5% (v/v) to about 2.0% (v/v).

A twenty first example includes the kit of the twentieth example, and includes at least two disposable volumetric pipettes.

A twenty second example includes the kit of the twentieth or twenty first examples, and comprises one or more microfuge tubes.

A twenty third example includes the kit of any one of the twentieth through twenty second examples, and further includes at least one assay control.

A twenty fourth example 24 includes the kit of any one of the twentieth through twenty third examples, and further specifies that the second processing chamber includes a plurality of reagents comprising a PLA reagent.

A twenty fifth example includes the kit of any one of the twentieth through twenty fourth examples, and further specifies that the PLA reagent is lyophilized.

A twenty sixth example includes the kit of any one of the twentieth through twenty fifth examples, and further specifies that the lateral flow device further comprises an amplification chamber in fluid connection with the first and/or second processing chamber.

A twenty seventh example includes the kit of the twenty sixth example, and further specifies that the amplification chambers comprise reagents necessary for an amplification process.

A twenty eighth example includes the kit of any one of the twentieth through twenty seventh examples, and further specifies that the lateral flow device further comprises a labeling chamber in fluidic connection with the first processing chamber, the second processing chamber, and/or the amplification chamber.

A twenty ninth example includes the kit of the twenty eighth example, and further specifies that the labeling chamber comprises a labeling agent.

A thirtieth example includes the kit of the twenty ninth example, and further specifies that the labeling agent comprises a fluorophore.

A thirty first example includes the kit of either the twenty ninth or thirtieth examples, and further specifies that the labeling agent is dried or lyophilized.

A thirty second example includes the kit of any one of the twentieth through the thirty first examples, and further specifies that the detection chamber comprises a lateral flow substrate.

A thirty third example includes the kit of the thirty second example, and further specifies that the lateral flow substrate comprises a binding moiety specific for at least a portion of the IP-10 target-linked oligonucleotide or amplicons thereof.

A thirty fourth example includes the kit of any one of the twentieth through the thirty third examples, and further specifies that the DMSO is in a range between about 0.5% (v/v) to about 1.5% (v/v).

In a thirty fifth example, a method for detection of a presence of IP-10 in a sample comprises introducing said sample into a lateral flow device comprising a plurality of chambers and a lateral flow substrate, said sample optionally treated with an aqueous solution containing dimethyl sulfoxide (DMSO) in a range between about 0.5% (v/v) to about 2.0% (v/v); contacting the sample in a first chamber of said plurality of chambers with an antibody-conjugate reagent that is specific for IP-10 or a fragment thereof, to thereby generate an analyte including a nucleic acid sequence comprised in said antibody-conjugate reagent; labeling said analyte in a second chamber of said plurality of chambers using a labeling reagent to generate a labeled analyte; and detecting a signal upon or after flowing said labeled analyte through said lateral flow substrate, said signal indicative of said presence of IP-10 in said sample.

A thirty sixth example includes the method of the thirty fifth example, and further specifies that the analyte further comprises the IP-10 or the fragment thereof.

A thirty seventh example includes the method of either of the thirty fifth or the thirty sixth examples, and further includes subjecting the analyte to an amplification process to generate an amplicon of the nucleic acid sequence.

A thirty eighth example includes the method of the thirty seventh example, and further specifies that the amplification process comprises PCR or an isothermal amplification process.

A thirty ninth example includes the method of any one of the thirty fifth through the thirty eighth examples, and further specifies that the labeling reagent comprises a fluorophore.

A fortieth example includes the method of any one of any one of the thirty seventh through the thirty ninth examples, and further specifies that the labeling is subsequent to the amplification process.

A forty first example includes the method of any one of the thirty fifth through the fortieth examples, and further specifies that the signal is an optical signal.

A forty second example includes the method of any one of the thirty fifth through the forty first examples, and further specifies that the antibody-conjugate reagent is a PLA reagent.

A forty third example includes the method of any one of the thirty fifth through the forty second examples, and further specifies that the signal is detected within 90 minutes of the sample being introduced into the lateral flow device.

A forth fourth example includes the method of any one of the thirty fifth through the forty third examples, and further specifies that the signal is detected within 45 minutes of the sample being introduced into the lateral flow device.

A forty fifth example includes the method of any one of the thirty fifth through the forty fourth examples, and further specifies that the sample is obtained from a subject having or suspected of having a disease.

A forty sixth example includes the method of any one of the thirty fifth through the forty fifth examples, and further specifies that the sample is a liquid sample.

In a forty seventh example, a method for detection of IP-10 in a sample comprises introducing said sample into a lateral flow device comprising a plurality of chambers and a lateral flow substrate, said sample having a volume of between 25 microliters (μL) and 100 μL; contacting said sample with an antibody-conjugate reagent specific for IP-10 or a fragment thereof in a first chamber of said plurality of chambers, to thereby generate an IP-10 target-linked oligonucleotide; directing said IP-10 target-linked oligonucleotide or amplicons thereof to flow through said lateral flow substrate; and detecting from said lateral flow substrate a signal that in response to a presence of IP-10 in said sample.

A forty eighth example includes the method of the forty seventh example, and further includes subjecting the IP-10 target-linked oligonucleotide to an amplification process to generate amplicons of the IP-10 target-linked oligonucleotide.

A forty ninth example includes the method of the forty eighth example, and further specifies that the amplification process includes PCR or an isothermal amplification process.

A fiftieth example includes the method of any one of the forty eighth or the forty ninth examples, and further includes contacting the IP-10 target-linked oligonucleotide or amplicons thereof with a labeling reagent to generate labeled IP-10 target-linked oligonucleotide or amplicons thereof.

A fifty first example includes the method of the fiftieth example, and further specifies that the labeling reagent comprises a fluorophore.

A fifty second example includes the method of the fiftieth or the fifty first examples, and further specifies that the directing the IP-10 target-linked oligonucleotide or amplicons thereof to flow through said lateral flow substrate comprises directing the labeled IP-10 target-linked oligonucleotide or amplicons thereof to flow through said lateral flow substrate.

A fifty third example includes the method of any one of the forty seventh through the fifty second examples, and further specifies that the signal is an optical signal.

A fifty fourth example includes the method of any one of the forty seventh through the fifty second examples, and further specifies that the sample is obtained from a subject having or suspected of having a disease.

A fifty fifth example includes the method of the fifty fourth example, and further specifies that the disease is a renal disease.

A fifty sixth example includes the method of the fifty fourth example, and further specifies that the disease is a renal disease.

A fifty seventh example includes the method of any one of the fifty fourth through the fifty sixth examples, and further includes determining a likelihood that the subject is having the disease.

A fifty eighth example includes the method of any one of the forty seventh through the fifty seventh examples, and further specifies that the sample has a volume between 50 μL and 70 μL.

Although the disclosure has been described in detail with particular reference to the described examples, other examples can achieve the same or similar results. The specification and examples are to be considered exemplary only and are not limiting to the claims.

1.-58. (canceled)

59. A cassette for detecting IP-10 in a sample, comprising:
a sample receiving zone configured to receive the sample comprising or suspected of comprising IP-10;
a plurality of chambers in fluid communication with one another;
one or more reagents disposed in at least one of the plurality of chambers, the one or more reagents comprising a PLA reagent which comprises:
a ligase configured to generate an IP-10-dependent oligonucleotide;
a polymerase configured to amplify of an IP-10-dependent oligonucleotide; and
a lateral flow substrate comprising a binding moiety specific for at least a portion of the IP-10-dependent oligonucleotide or amplicons thereof.

60. The cassette of claim 59, wherein the plurality of chambers comprises a sample chamber which comprises the sample receiving zone.

61. The cassette of claim 59, wherein the one or more reagents comprise dried or lyophilized reagents.

62. The cassette of claim 59, wherein the plurality of chambers further comprises at least a first chamber in fluid communication with the sample chamber, and a second chamber in fluid communication with the first chamber.

63. The cassette of claim 62, further comprising at least a first reagent recess and a second reagent recess.

64. The cassette of claim 63, wherein the first reagent recess is in fluid communication with and proximal to an outlet of the sample chamber and/or an inlet of the first chamber.

65. The cassette of claim 64, wherein the one or more reagents further comprise a sample pretreatment reagent or an antibody conjugate reagent.

66. The cassette of claim 65, wherein the antibody conjugate reagent comprises two antibody conjugates, each coupled to complementary oligonucleotide strands and each directed to a different epitope of IP-10.

67. The cassette of claim 65, wherein the sample pretreatment reagent or the antibody conjugate reagent is disposed in the first reagent recess.

68. The cassette of claim 63, wherein the second reagent recess is in fluid communication with and proximal to an outlet of the first chamber and/or an inlet of the second chamber.

69. The cassette of claim 67, wherein the PLA reagent is disposed in the second reagent recess.

70. The cassette of claim 59, wherein the plurality of chambers comprises a detection chamber for detecting the present or absence of the IP-10-dependent oligonucleotide or amplicons thereof, the detection chamber comprising the lateral flow substrate.

71. The cassette of claim 70, wherein the detection chamber further comprises a fluid compartment containing detection particles for labeling IP-10-dependent oligonucleotide or amplicons thereof.

72. The cassette of claim 59, further comprising a plurality of vents in connection with said plurality of chambers.

73. The cassette of claim 59, wherein the cassette is disposable.

74. A kit for performing point of care testing, comprising:
a lateral flow device configured for assaying a sample comprising or suspected of comprising IP-10, the lateral flow device comprising:
a processing chamber configured for generating IP-10-dependent oligonucleotide or amplicons thereof;
a detection chamber for detection the presence or absence of the IP-10-dependent oligonucleotide or amplicons thereof; and
one or more reagents comprising a PLA reagent; and
one or more disposable volumetric pipettes.

75. The kit of claim 74, further comprising one or more microfuge tubes.

76. The kit of claim 74, further comprising at least one assay control.

77. The kit of claim 74, further comprising a buffer containing dimethyl sulfoxide (DMSO) in a range between about 0.5% (v/v) to about 2.0% (v/v).

78. The kit of claim 74, wherein the detection chamber comprises a lateral flow substrate.
